(19) United States
(12) Patent Application Publication
(10) Pub. No.: US 2009/0131752 A1
(43) Pub. Date: May 21, 2009

(54) INFLATABLE ARTIFICIAL MUSCLE FOR ELONGATED INSTRUMENT

(75) Inventor: Chul Hi Park, Los Altos, CA (US)

Correspondence Address: AMPACC LAW GROUP, 13024 Beverly Park Road, Suite 205, Mukilteo, WA, 98275, US

(73) Assignee: Chul Hi Park, Los Altos, CA (US)

(21) Appl. No.: 12/271,902

(22) Filed: Nov. 16, 2008

Related U.S. Application Data
(60) Provisional application No. 60/989,074, filed on Nov. 19, 2007.

(51) Int. Cl. A61B 1/00

(52) U.S. Cl. 600/114

(57) ABSTRACT

An inflatable artificial muscle for manipulating a flexible elongated instrument such as a medical endoscope that advantageously utilizes fluid pressure to facilitate insertion into a tortuous passage and maneuver of the instrument is described herein. The inflatable artificial muscle comprises a hollow cylinder-like bladder with a lumen or a plurality of such bladders joined end to end for receiving an elongated instrument. The bladder volume is divided into a plurality of chambers of varying configurations, which are in fluid communications with one another. There is provided a plurality of supply tubes for supplying and evacuating a pressurizing fluid to and from the bladder. The inflatable artificial muscle is activated by inflating the bladder with a pressurizing fluid to a predetermined internal pressure. The artificial muscle of the present invention is capable of straightening a portion of an elongated instrument in a bent configuration, which is enclosed within the lumen thereof.

### BACKGROUND OF THE INVENTION

The present invention relates generally to an inflatable artificial muscle for manipulating a flexible elongated instrument. More particularly, it relates to an inflatable artificial muscle that is operated, in part, by fluid pressure to facilitate insertion of a flexible elongated instrument, such as a medical endoscope, into a tortuous passage and maneuver the instrument therein for diagnostics, examination and treatment in medical and industrial applications.

A flexible endoscope is an instrument having an elongated tubular shape for viewing the interior of a passage, for example, an organ of a patient such as the colon or a conduit in industrial equipment. Flexible endoscopes can be used for a variety of different diagnostic and interventional procedures in medical applications, including colonoscopy, sigmoidoscopy, bronchoscopy, thoracoscopy, laparoscopy and video endoscopy, and examination or inspection of interior parts of industrial hardware such as a jet engine that are hard to gain access to and visualize with an ordinary inspection instrument.

Typically equipped with a visualization means, such as a miniature video camera, at the leading end, the frontal tip of flexible endoscope is usually advanced into the region to be examined by a pushing force provided by an operator from outside the passage where the endoscope is being deployed. A distinctive advantage of flexible endoscopes over rigid ones is that their flexible bodies allow them to readily conform to a complex geometry of a passage that may be difficult or impossible to navigate with a rigid and, often, straight endoscopes. The pliable nature of the body of flexible endoscope can also be a disadvantage in some practical applications making the advancement of the instrument along a passage challenging. The most serious difficulties are encountered when an operator tries to maneuver a flexible endoscope into a passage that is highly tortuous and mobile and/or more pliable and distensible than the body of the endoscope. A prominent example of such mobile and tortuous passage is the colon of a patient undergoing a colonoscopy procedure.

Colonoscopy is a medical procedure in which a flexible endoscope, or colonoscope, is inserted into patient's colon for diagnostic examination or surgical treatment of the colon. A standard colonoscope is typically 135-185 cm in length and 8-19 mm in diameter. It may include a fiber-optic imaging bundle or a miniature camera located at the instrument's tip, illumination fibers, one or two instrument channels that may also be used for insufflation or irrigation, air and water channels, and vacuum channels. The most commonly used procedure for examining the colon is to insert a colonoscope as far into the colon as desired while inspecting as the colonoscope advances. A detailed examination of the colon is made as the colonoscope is withdrawn. To examine the entire colon, the colonoscope is inserted through the anus into the rectum, and then advanced through the sigmoid colon into the descending colon. The colonoscope then passes through the left colic flexure (the splenic flexure) into the transverse colon, and then through the right colic flexure (the hepatic flexure). The colonoscope next passes through the ascending colon and finally reaches the cecum.

Located adjacent to the rectum, the sigmoid colon is the most tortuous part of a patient's colon with a number of acute bends and of highly convoluted configuration sharing small space in the pelvic cavity with other organs. Repeated application of steering and advancing maneuvers by an endoscopist in the sigmoid colon often leaves a crooked and/or a loop-like formation in the shaft of flexible body of colonoscope. This formation tends to be enlarged whenever the advancing portion of shaft or the tip of endoscope is confronted with an obstacle upstream in the colon such as sharp bends in the splenic or hepatic flexures or other form of resistance such as friction between the shaft and the colon wall. If not dealt with, the enlargement of these formations are bound to become a main cause of patient pain and serious difficulties in advancing the instrument into deeper part of the colon.

An endoscopist often devotes a considerable amount of procedure time and efforts to undo and straighten the undesirable formation in the shaft of endoscope throughout colonoscopy procedure. The maneuvers required to accomplish this generally demand high level of skills from an endoscopist and frequently cause severe discomforts to the patient.

Devising means to control the body of a flexible elongated instrument such as endoscopes presents significant technical challenges. Most flexible endoscopes are generally constructed out of a large number of short, rigid segments interconnected serially with neighboring segments by a pair of joints with, typically, one or two degrees of freedom. A complete control of such an instrument entails abilities to independently manipulate every joint or segment in an arbitrary manner, which may require a device with an inordinately complex functionality and is likely impractical.

Belson (U.S. Pat. No. 6,610,007, which is incorporated by reference) discloses an endoscope instrument, a portion of which is automatically controllable. Motors are provided for all the segments in the controllable portion of the instrument to independently control each segment. The resulting instrument is highly complex and susceptible to mechanical failure.

Levy (U.S. Pat. App. No. 20060183974, which is incorporated by reference) discloses an endoscope with an insertion tube fitted with an optical head. The insertion tube is coupled with a major inflatable sleeve and auxiliary inflatable sleeves, which upon inflation is capable of propelling the endoscope within the conduit. These inflatable sleeves are not capable of actively manipulating the insertion tube of the endoscope.

Belson (U.S. Pat. App. No. 20060258912, which is incorporated by reference) discloses a segmented, elongated endoscope instrument, a portion of which can be articulated by electro-polymeric materials. Adjacent segments are articulated by inducing relative differences in size or length of the material placed around the periphery of the instrument using the electro-polymeric materials, which are configured to contract or expand under a stimulus. This method of actuation is applicable to a purposely constructed endoscope instrument but not to an ordinary instrument.

Bauerfeind (U.S. Pat. No. 5,337,733, which is incorporated by reference) discloses an insertion aid device for colonoscope of tubular configuration that can be converted between rigid and flexible states through the use of fluid pressure. The device is a passive device useful for guiding a colonoscope and does not have capabilities to actively manipulate the shaft of colonoscope. Mouris-Laan (U.S. Pat. No. 5,882,347, which is incorporated by reference) discloses a medical catheter of construction similar to that of the insertion aid device of Bauerfeind's '733 patent.

U.S. patent application Ser. Nos. 11/872,025 and 12/113,073 (assigned to this inventor), which are incorporated by reference, describe inflatable actuation devices that can be collapsed into a small profile for easy introduction into the colon through the anus and for actively manipulating the shaft of endoscope during insertion into a patient's colon.

There is a need to provide an improved device for inserting an instrument into a living organ having a tortuous passage for medical diagnostics, examination and/or treatment.

### BRIEF SUMMARY OF THE INVENTION

For the purposes of this disclosure, including the appended claims, the terms “distal”, “distally”, and “distal end”, as they relate to the devices and methods described herein, refer to the end of the device further from or in the direction away from an operator who might be applying the device or method to the subject. Stated otherwise, the terms refer to the end of the device closer to or in the direction towards the patient's interior.

The terms “proximal”, “proximally”, and “proximal end”, as they relate to the devices and methods described herein, refer to the end of the device closer to or in the direction towards the operator who might be applying the device or method, rather than the patient.

In one embodiment, an inflatable artificial muscle comprises a substantially hollow cylinder-like bladder with a lumen therein comprising a shaped inner sleeve bounding the lumen and a shaped, outer sleeve and a plurality of supply tubes. The inflatable artificial muscle receives a flexible elongated instrument in a bent configuration through the lumen of the bladder and straightens it when activated by pressurizing fluid supplied through the supply tubes from a source external to the passage such as the colon where the flexible elongated instrument is deployed for, e.g., medical examination. The inflatable artificial muscle is deactivated by evacuating the pressurizing fluid from the bladder. In one implementation, the inflatable artificial muscle may be used to maintain a portion of a flexible elongated instrument in a substantially straightened configuration. In another implementation, an inflatable artificial muscle of the present invention may be constructed by joining a plurality of hollow cylinder-like bladders end to end and in fluid communications with one another. Although the embodiments and implementations described in this specification specifically refer to the colon and colonoscopy procedure, the scope of the present invention applicability is not limited to the colon or any particular bodily organ.

In another embodiment, a substantially hollow cylinder-like bladder (also referred to herein after as, “bladder”) comprising an inflatable artificial muscle may be divided into a plurality of interconnected chambers that are in fluid communications with one another and bounded by a plurality of baffle-like partitions that are parts of and built into the shaped inner sleeve. In one implementation, a plurality of the partitions may be arranged in parallel with the general axis of the bladder creating a plurality of axial chambers in the bladder volume. In another implementation, a plurality of the partitions may be arranged around the circumference of the shaped inner sleeve to be substantially perpendicular to the general axis thereof creating a plurality of radial chambers. In another implementation, the radial chambers created by a plurality of circumferential partitions may be further divided into a plurality of axial chambers by a plurality of axial partitions disposed between neighboring radial partitions. In yet another implementation, a plurality of the partitions may be arranged at oblique angles with respect to the general axis of the bladder creating a plurality of irregularly shaped chambers. In yet another implementation, the shaped inner sleeve and shaped outer sleeve may be fixedly joined at predetermined locations along the outer most edges of partitions in the shaped inner sleeve.

### BRIEF DESCRIPTION OF THE DRAWINGS

The following exemplary figures are provided to supplement the description below and more clearly describe the invention. In the figures, like elements are generally designated with the same reference numeral for illustrative convenience and should not be used to limit the scope of the present invention.

FIG. 1A is a schematic, perspective view of an inflatable artificial muscle in a fully expanded state according to an embodiment of the present invention.

FIG. 1B is a sectional view taken along the line 1B-1B in FIG. 1A according to an embodiment of the present invention.

FIG. 1C is a schematic, perspective view of an inflatable artificial muscle in a fully expanded state according to another embodiment of the present invention.

FIG. 2A is a schematic, perspective view of a shaped outer sleeve in a fully expanded state according to an embodiment of the present invention.

FIG. 2B is a sectional view taken along the line 2B-2B of a shaped outer sleeve in a fully expanded state, shown in FIG. 2A, according to an embodiment of the present invention.

FIG. 2C is a schematic, perspective view of a shaped outer sleeve in a fully expanded state according to another embodiment of the present invention.

FIG. 3A is a schematic, perspective view of a shaped inner sleeve according to an embodiment of the present invention.

FIG. 3B is a sectional view taken along the line 3B-3B of the shaped inner sleeve, shown in FIG. 3A, according to an embodiment of the present invention.

FIG. 4 is a schematic, perspective view of a hollow cylinder-like bladder in an expanded state with a portion of the shaped outer sleeve removed according to an embodiment of the present invention.

FIG. 5A is a schematic, perspective view of a shaped inner sleeve according to another embodiment of the present invention.

FIG. 5B is a sectional view taken along the line 5B-5B of the shaped inner sleeve, shown in FIG. 5A, according to an embodiment of the present invention.

FIG. 6A is a schematic, perspective view of a hollow cylinder-like bladder in an expanded state with a portion of the shaped outer sleeve removed according to another embodiment of the present invention.

FIG. 6B is a sectional view taken along the line 6B-6B of the shaped inner sleeve, shown in FIG. 6A, according to an embodiment of the present invention.

FIGS. 7A and 7B are sectional views showing variations in the shaped inner sleeve with axial partitions according to embodiments of the present invention.

FIGS. 8A, 8B and 9 are schematic, perspective views of a shaped inner sleeve according to further embodiments of the present invention.

### DETAILED DESCRIPTION OF THE INVENTION

Embodiments of the present invention relate to an inflatable artificial muscle that utilizes fluid pressure to manipulate a portion of a flexible elongated instrument, for example, a medical endoscope.

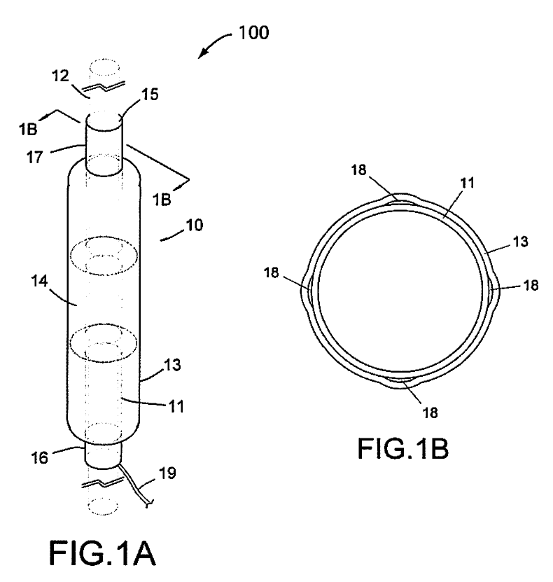

Referring to FIG. 1A, an inflatable artificial muscle 100 (or inflatable device) is schematically shown mounted on the shaft of a flexible elongated instrument 12 according to one embodiment of the present invention. Inflatable artificial muscle 100 includes a bladder 10 and a lumen 15. Bladder 10 has a hollow cylinder-like shape and is shown in an expanded state. Lumen 15 includes a shaped inner sleeve 11 bounding lumen 15 and a shaped, outer sleeve 13, which together define a bladder volume 14 of generally hollow cylinder-like shape, and a plurality of supply tubes for supplying and evacuating a pressurizing fluid to and from bladder volume 14. In one implementation, shaped inner 11 and outer 13 sleeves are sealingly joined circumferentially, except where a plurality of supply tubes are disposed in case they are integrated with the sealing joint, in proximal and distal portions 16, 17 (also referred to hereinafter as, “neck”) to form a fluid tight envelope of bladder volume 14. A plurality of supply tubes 19 is provided for supplying a pressurizing fluid to the hollow cylinder-like bladder from a source outside the passage where the flexible elongated instrument is deployed and evacuating the pressurizing fluid from the bladder. In one implementation, the supply tubes may be integrated with the sealing joint at the neck locations. In another implementation, the supply tubes may be embedded in or integrated with shaped inner sleeve 11 with a plurality of outlets disposed at predetermined locations along the length thereof. A plurality of the hollow cylinder-like bladders 101, 102 may be joined in a series to construct an elongated inflatable artificial muscle of a predetermined length, as shown in FIG. 1C. In this case fluid-tight seals with provisions for the supply tubes 103 are made between the shaped inner and outer sleeves at the proximal and distal most neck portions and in the intervening neck locations to allow for fluid communication between neighboring hollow cylinder-like bladders. FIG. 1B shows an exemplary embodiment of a cross section, taken along the line 1B-1B in FIG. 1A, of a partially joined neck where open fluid channels 18 are disposed at predetermined circumferential locations between shaped inner 11 and outer 13 sleeves.

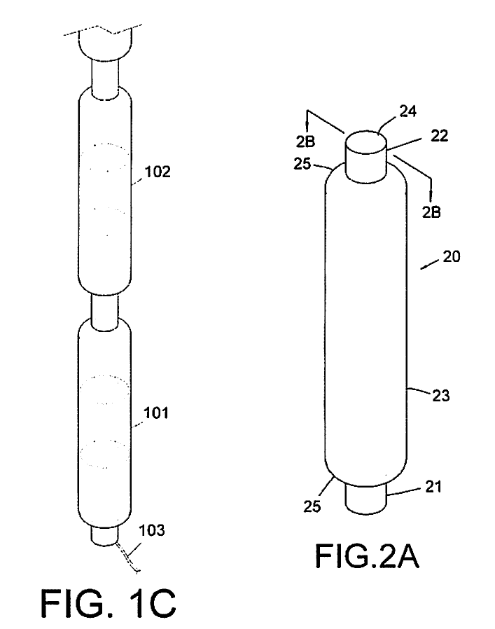

Referring to FIG. 2A, the shaped outer sleeve 20 is of a shaped cylinder configuration with a lumen 24, shoulder portions 25, proximal and distal necks or ends 21, 22 and body portion 23 of substantially cylindrical symmetry in an embodiment of the present invention. In another embodiment, body portion 23 may be of a barrel shape with a larger diameter near the midsection thereof than the rest of the body, as shown in FIG. 2C. FIG. 2B shows a sectional view of shaped outer sleeve 20 taken along the line 2B-2B in FIG. 2A showing lumen 24, proximal and distal necks 21, 22, and shoulder portions 25 in detail. The length and diameter at the midsection of the shaped outer sleeve may be chosen to suit a particular application and in consideration of the geometrical constraint imposed by the passage where the inflatable artificial muscle is deployed. In the case of the colon, the length may be between 4 cm and 20 cm and the diameter between 1.5 cm and 5 cm.

The construction material for the shaped outer sleeve may be flexible and substantially non-compliant or semi-compliant. A non-compliant material is generally stiff and resists stretching and maintains a design length or a design shape even when acted on by a force not exceeding a given magnitude. A semi-compliant material is not as stiff as a non-compliant material but able to resist stretching and maintains a design length or a design shape even when acted on by a force of a moderate magnitude. A bladder comprising sleeves of non-compliant materials is able to withstand internal pressure in a fully inflated state and maintain its design shape without expanding freely in proportion to the internal pressure, as a latex balloon would, until it ruptures. These materials may be soft and thin enough to allow the shaped outer sleeve to be pleated into a low profile form when the hollow cylinder-like bladder comprising the inflatable artificial muscle is in a deflated state. Suitable materials includes thermoplastic film material such as polyethylene terephthalate (PET), polypropylene, polyamide (Nylon), polyimide (Kapton), polyvinylchloride (PVC), polyurethane, Pebax and polyethylene, of various grades. Alternately, latex or silicon rubber material of suitable dimensions may be employed for the construction. Alternately, the shaped outer sleeve may be made of a shaped tube reinforced with an embedded mesh of resilient material. Any number of methods for joining or bonding shaped inner and outer sleeves may be employed. For example, a thermal bonding or a bonding method based on an adhesive, or a combination of both may be utilized. An intermediate polymer layer such as ethylene-vinyl acetate (EVA) may be used for a thermal bonding to enhance the bonding strength. A predetermined mechanical joining method may also be employed to provide a bonding as well as a means for relieving strain on the joint. A blow molding, a thermo-forming or a number of other methods well known to the art of shaping a thin film polymeric material may be employed to construct the shaped outer sleeve. Any low viscosity fluid may be used to inflate the hollow cylinder-like bladder, for example, air, carbon dioxide, water and a saline solution.

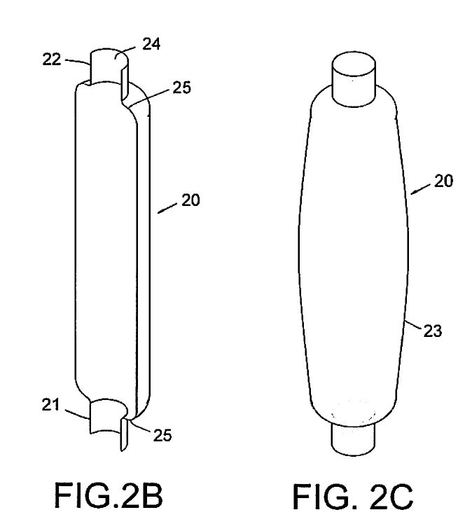

Referring to FIG. 3A, the shaped inner sleeve 30 is generally of cylindrical configuration with a lumen 34, proximal and distal necks or ends 33, 34 and a plurality of radial ridge-like or baffle-like portions of flattened torus-like shape 31, which will be referred to as radial partition hereinafter, disposed substantially perpendicularly to the general axis and projecting outwardly with respect to lumen 34 at predetermined positions along the length thereof in an embodiment of the present invention. FIG. 3B is a sectional view of shaped inner sleeve 30 taken along the line 3B-3B in FIG. 3A showing details of the configuration of radial partition 31 and lumen 34. The length of the shaped inner sleeve may be chosen to be substantially comparable to those of the body and shoulder portions of the shaped outer sleeve combined. The diameter of lumen 34 may be chosen so that the elongated instrument, where the inflatable artificial muscle is used, may be accommodated snugly therein, for example, 0.7 cm and 2 cm. The diameter of radial partitions may be varied to be slightly smaller than that of the body and shoulder portions of the shaped outer sleeve, respectively. The flap-like geometry of radial partitions allows them to be readily folded flat around the cylindrical body of the shaped inner sleeve, which facilitates the introduction of the inflatable artificial muscle into a passage through a constricted access port, such as when a medical endoscope is introduced into the colon through the anus.

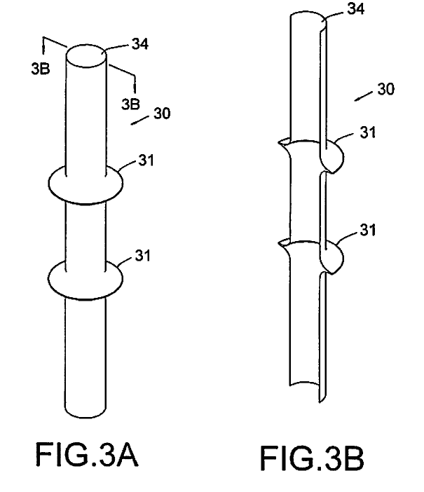

FIG. 4 shows an embodiment of hollow-cylinder like bladder 40 comprising the inflatable artificial muscle of the present invention where part of shaped outer sleeve 13 is removed to show the details of shaped inner sleeve 30 with radial partitions 31 and its disposition within shaped outer sleeve 13. In one implementation, the bladder volume 42 is divided into a plurality of interconnected hollow cylinder-like radial chambers 43 by a plurality of radial partitions 31. In an implementation, shaped inner 30 and outer 13 sleeves may be fixedly joined at predetermined locations along the outermost edges of radial partitions 31.

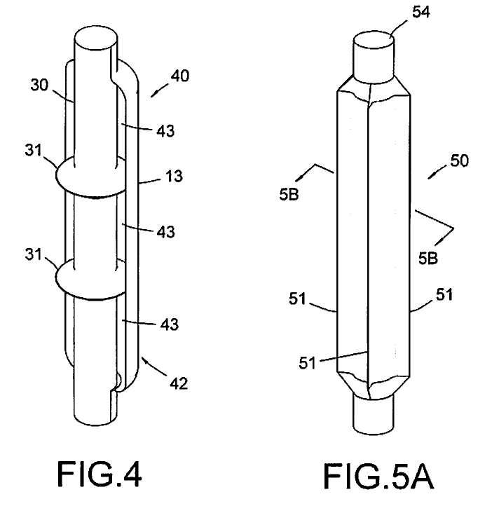

In another embodiment as schematically shown in FIG. 5A, the shaped inner sleeve 50 includes a lumen 54 and a plurality of axial ridge-like portions 51, which will be referred to hereinafter as, “axial partition,” disposed substantially parallel to the general axis at predetermined positions around the circumference thereof. FIG. 5B is a sectional view taken along the line 5B-5B of shaped inner sleeve 50 shown in FIG. 5A showing details of axial partitions 51. The flap-like geometry of axial partitions allows them to be readily folded flat onto the cylindrical body of the shaped inner sleeve for easy introduction of the inflatable artificial muscle into a passage.

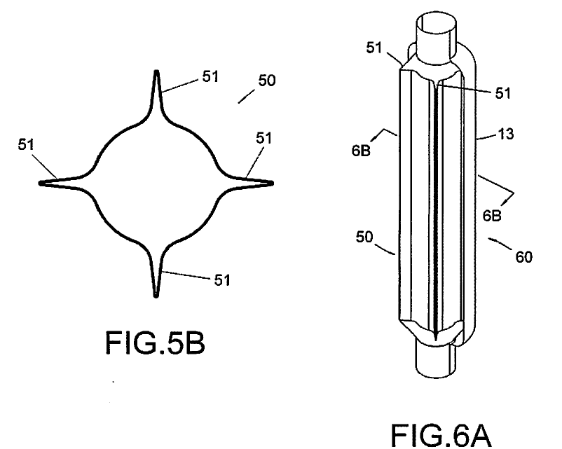

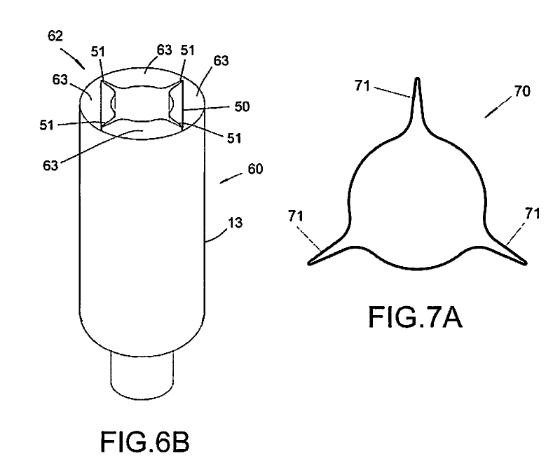

FIG. 6A shows an embodiment of hollow-cylinder like bladder 60 comprising the artificial muscle of the present invention where part of shaped outer sleeve 13 is removed to show the details of shaped inner sleeve 50 with axial partitions 51 and its disposition within shaped outer sleeve 13. FIG. 6B is a sectional view taken along the line 6B-6B in FIG. 6A of hollow-cylinder like bladder 60 comprising shaped outer sleeve 13 and shaped inner sleeve 50. In one implementation, the bladder volume 62 is divided into a plurality of interconnected axial chambers 63 substantially parallel to the general axis thereof by a plurality of axial partitions 51. The radial extension of an axial partition may be varied to be slightly smaller than the radius of the body and shoulder portions of shaped outer sleeve, respectively, so that neighboring axial chambers may be in fluid communications with each other. As shown in FIGS. 7A and 7B in exemplary embodiments, any number of axial partitions 71 may be included in the shaped inner sleeve 70. In an implementation, inner 50 and outer 13 sleeves may be fixedly joined at predetermined locations along the outermost edges of axial partitions 51.

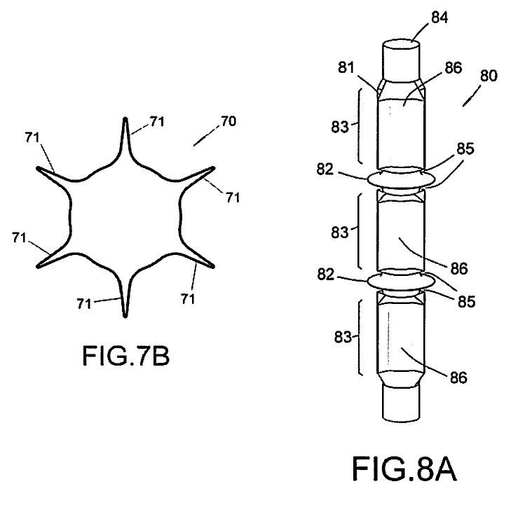

Referring to FIGS. 8A and 8B, a shaped inner sleeve 80 includes a plurality of partition portions 83 according to another embodiment of the present invention. Each partition portion 83 includes a plurality of axial partitions 81 and a plurality of radial partitions 82. Shaped inner sleeve 80 may include gaps 85 between axial 81 and radial 82 partitions. Gaps 85 allow axial and radial partitions to be folded onto the body of the shaped inner sleeve more readily for easy introduction of the inflatable artificial muscle into a passage. In another implementation, axial 81 and radial 82 partitions may be merged to form a continuous ridge-like configuration 87 of a predetermined geometric pattern. The bladder volume of the inflatable artificial muscle comprising the present embodiment of shaped inner sleeve and a shaped outer sleeve (not shown) is divided into a plurality of interconnected chambers 86 bounded by axial and radial partitions on four sides. Shaped inner sleeve may be fixedly joined with shaped outer sleeve at predetermined axial locations along the outermost edges of ridge-like partitions.

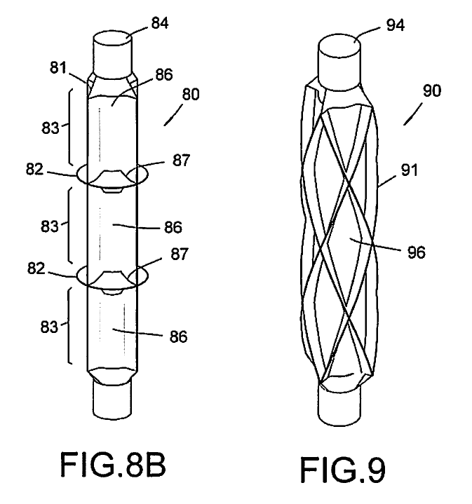

Referring to FIG. 9, shaped inner sleeve 90 includes a lumen 94 and a plurality of ridge-like partitions 91 at oblique angles with respect to the general axis thereof according to another embodiment of the present invention. The bladder volume comprising of shaped inner sleeve and a shaped outer sleeve (not shown), such as the one shown in FIG. 2A, is divided into a plurality of interconnected chambers 96 bounded by partitions of shaped inner sleeve on four sides. The radial extension of the partition may be varied to be slightly smaller than the radius of the body and shoulder portions of the shaped outer sleeve, respectively, so that neighboring chambers may be in fluid communication with one another. The shaped inner sleeve may be fixedly joined with the shaped outer sleeve at predetermined locations along the outermost edges of ridge-like partitions.

The construction material for the shaped inner sleeve may be flexible and substantially non-compliant or semi-compliant. This material preferably should be soft and thin enough to allow the inner sleeve to be pleated into a low profile form when the hollow cylinder-like bladder comprising the inflatable artificial muscle is in a deflated state. Suitable materials includes thermoplastic film material such as polyethylene terephthalate (PET), polypropylene, polyamide (Nylon), polyimide (Kapton), polyvinylchloride (PVC), polyurethane, Pebax and polyethylene, of various grades. Alternately, latex or silicon rubber material of predetermined dimensions may be employed for the construction. Alternately, the shaped inner sleeve may be made of a shaped tube reinforced with an embedded mesh of resilient material. A blow molding, a thermo-forming or a number of other methods well known to the art of shaping a thin film polymeric material may be employed to construct the shaped inner sleeve.

The inflatable artificial muscle of the present invention is capable of providing a restoring force to straighten a portion of the body of a flexible elongated instrument enclosed therein in a bent configuration. The inflatable artificial muscle may be used to maintain a portion of a flexible elongated instrument in a substantially straightened configuration taking advantage of its ability to resist deformation under external load. The artificial muscle is activated or energized by the internal pressure built up in the hollow cylinder-like bladder by a pressurizing fluid supplied through supply tubes from sources external to the passage where the instrument is deployed. The artificial muscle is deactivated by deflating or evacuating the pressurizing fluid from the hollow cylinder-like bladder. The restoring force is primarily provided by the tensile stress induced on the shaped outer sleeve comprising the hollow cylinder-like bladder by the internal pressure force. The extent of recovery from a bent to straight configuration is determined by relative magnitudes of tensile stresses contributed by the internal pressure and the external bending loads, which are a combination of loads exerted on the artificial muscle and the elongated instrument. The mechanical behavior of a cylindrical inflatable vessel under internal pressure and external loads is well known to those familiar with the art of inflatable structures (for example, see J. D. Suhey, N. H. Kim, C. Neizrecki, “Numerical modeling and design of inflatable structures-application to open-ocean-aquaculture cages.”, Aquacultural Engineering 33 (2005), 285-303, which is incorporated by reference). The magnitude of restoring force provided by the inflatable artificial muscle depends on the internal pressure and more strongly, on the geometry and overall dimensions of the shaped outer sleeve. For application in the colonoscopy, a suitable range of the magnitude of restoring force may be between 2 Newton (or N) to 35 N, preferably, 5 N and 20 N. The operating pressure of the present actuation device may be between 0.1 atm and 8 atm, preferably. 0.5 atm and 5 atm.

The chambers formed in the bladder volume between the shaped inner and outer sleeves are in such a size and configuration that they can better resist deformation under external loads exerted by a flexible elongated instrument in a bent configuration than a bladder without such partitions. The restoring force of the shaped outer sleeve is transferred to the shaped inner sleeve, which is in direct contact with the elongated instrument, through the internal pressure built up by pressurizing fluid in these chambers. The bent elongated instrument works to compress and deform the chambers by exerting compressive forces on the portion of the shaped inner sleeve that forms a part of chamber walls. The tensile stress induced on the partitions comprising the chambers and portions of the shaped inner sleeve in contact with the elongated instrument works against the load from the elongated instrument. While the magnitude of the restoring force transferred to the elongated instrument through the shaped inner sleeve is larger than the combined load that brought the elongated instrument to a bent configuration, the straightening of the elongated instrument by the inflatable artificial muscle continues. The straightening stops when these two opposing forces balance each other.

While preferred illustrative embodiments of the invention are described above, it will be apparent to those skilled in the art that various changes and modifications may be made therein without departing from the invention. Accordingly, the appended claims should be used to interpret the scope of the present invention.

What is claimed is:

1. An inflatable artificial muscle for manipulating a flexible elongated instrument, the inflatable artificial muscle comprising:
a shaped inner sleeve having a proximal end and a distal end, and a lumen;
a shaped outer sleeve having a body portion, a proximal end and a distal end; and
a plurality of supply tubes;
wherein said shaped inner sleeve includes a plurality of partition portions protruding outwardly with respect to said lumen thereof;
wherein said shaped inner sleeve is sealingly joined with said shaped outer sleeve at said proximal and distal ends and together form a hollow cylinder-like bladder having a lumen defined by said lumen of said shaped inner sleeve;
wherein said hollow cylinder-like bladder is divided into a plurality of chambers that are in fluid communications with one another;
wherein the boundaries of said chamber of said hollow cylinder-like bladder are defined by portions of said shaped inner sleeve and said outer sleeve and a plurality of the partition portions of said shaped inner sleeve;
wherein said plurality of supply tubes are configured to supply and evacuate a pressurizing fluid to and from said hollow cylinder-like bladder; and
wherein said hollow cylinder-like bladder is configured to receive a portion of a flexible elongated instrument and substantially straighten the portion of the flexible elongated instrument and maintain the portion of the flexible elongated instrument in a substantially straightened configuration when activated by a pressurizing fluid.

2. The inflatable artificial muscle of claim 1, wherein said body portion of said shaped outer sleeve is substantially of a cylinder shape.

3. The inflatable artificial muscle of claim 1, wherein said body portion of said shaped outer sleeve is substantially of a barrel shape.

4. The inflatable artificial muscle of claim 1, wherein said plurality of supply tubes are integrated with the sealing joint between said shaped inner sleeve and said shaped outer sleeve.

5. The inflatable artificial muscle of claim 1, wherein said plurality of supply tubes are embedded in said shaped inner sleeve with a plurality of outlets disposed along the length thereof.

6. The inflatable artificial muscle of claim 1, wherein said plurality of partition portions is disposed circumferentially around said shaped inner sleeve.

7. The inflatable artificial muscle of claim 1, wherein said plurality of partition portions is disposed along the length of said shaped inner sleeve.

8. The inflatable artificial muscle of claim 1, wherein said plurality of partition portions is disposed at predetermined angles with respect to one another.

9. The inflatable artificial muscle of claim 1, wherein said shaped inner sleeve and said shaped outer sleeve are fixedly joined along predetermined locations on edges of said partition portions of said shaped inner sleeve.

10. An inflatable device for manipulating a flexible elongated instrument, the inflatable device comprising:
a plurality of hollow cylinder-like bladders, each hollow cylinder-like bladder having a lumen, a proximal end and a distal end, said plurality of hollow cylinder-like bladders being serially joined end to end with said lumens thereof aligned with respect to one another to form an elongated lumen, said plurality of hollow cylinder-like bladders being in fluid communications with one another and with a plurality of supply tubes,
wherein said hollow cylinder-like bladder includes:
a shaped inner sleeve having a proximal end and a distal end, and a lumen, and
a shaped outer sleeve having a proximal end and a distal end.

11. The inflatable device of claim 10, wherein said shaped inner sleeve includes a plurality of partition portions protruding outwardly with respect to said lumen thereof;
wherein said shaped inner sleeve is sealingly joined with said shaped outer sleeve at said proximal and distal ends and together form said hollow cylinder-like bladder having a lumen defined by said lumen of said shaped inner sleeve;
wherein said hollow cylinder-like bladder is divided into a plurality of chambers that are in fluid communications with one another;
wherein the boundaries of said chamber of said hollow cylinder-like bladder are defined by portions of said shaped inner sleeve and said outer sleeve, and a plurality of the partition portions of said shaped inner sleeve; and
wherein said plurality of supply tubes are configured to supply and evacuate a pressurizing fluid to and from said hollow cylinder-like bladders; and
wherein each hollow cylinder-like bladder of said plurality of hollow cylinder-like bladders is configured to receive and substantially straighten a portion of the flexible elongated instrument and maintain the portion of the flexible elongated instrument in a substantially straightened configuration when activated by the pressurizing fluid.

12. The device of claim 10, wherein said hollow cylinder-like bladders have substantially identical dimensions.

13. The device of claim 10, wherein said hollow cylinder-like bladders have varying dimensions from one another.

14. An inflatable device for manipulating a flexible elongated instrument, the inflatable device comprising:
a shaped inner sleeve having a lumen, a proximal end and a distal end;
a shaped outer sleeve having a proximal end and a distal end; and
a plurality of supply tubes;
wherein said shaped inner sleeve is sealingly joined with said shaped outer sleeve at said proximal and distal ends and together form a hollow cylinder-like bladder having a lumen defined by said lumen of said shaped inner sleeve,
wherein said shaped inner sleeve includes a plurality of ridge-like portions projecting outwardly with respect to said lumen thereof and toward said shaped outer sleeve,
wherein said hollow cylinder-like bladder is divided into a plurality of chambers that are in fluid communications with one another,
wherein said chamber is bounded by a plurality of ridge-like portions of said shaped inner sleeve,
wherein said plurality of supply tubes are configured to supply and evacuate a pressurizing fluid to and from said hollow cylinder-like bladder, and
wherein said hollow cylinder-like bladder is configured to receive and substantially straighten a portion of a flexible elongated instrument and maintains the portion of the flexible elongated instrument in a substantially straightened configuration when activated by a pressurizing fluid.

15. The device of claim 14, wherein said shaped inner sleeve and said shaped outer sleeve are fixedly joined at predetermined locations along said plurality of ridge-like portions of said shaped inner sleeve.

16. The device of claim 14, wherein said plurality of ridge-like portions of said shaped inner sleeve is disposed separated from one another.

17. The device of claim 14, wherein said plurality of ridge-like portions of said shaped inner sleeve is disposed to intersect one another.
